(19) United States
(12) Patent Application Publication
(10) Pub. No.: US 2007/0203564 A1
(43) Pub. Date: Aug. 30, 2007

(54) Biodegradable implants having accelerated biodegradation properties in vivo

(75) Inventors: Emily E. Rusk, Boston, MA (US); F. Anthony Headley JR., Marlborough, MA (US); Claude Clerc, Marlborough, MA (US)

Correspondence Address: HOFFMANN & BARON, LLP, 6900 JERICHO TURNPIKE, SYOSSET, NY, 11791, US

(73) Assignee: Boston Scientific Scimed, Inc.

(21) Appl. No.: 11/363,836

(22) Filed: Feb. 28, 2006

(51) Int. Cl. A61F 2/82; H01J 37/30; H05B 6/00

(52) U.S. Cl. 623/1.13; 623/1.38; 623/23.75; 264/485; 264/488

(57) ABSTRACT

The present invention is directed to a biodegradable implant including a biodegradable polymer previously exposed to conditions of biodegradation such as chemical, thermal or radiation degradation. The present invention further includes the possibility of attaching axial runners to the implant. The present invention is further directed to a method of forming a biodegradable implant, such as a stent, by irradiation of the individual filaments or fibers, or irradiation of the formed implant.

### FIELD OF THE INVENTION

The present invention relates to biodegradable implantable prostheses having accelerated biodegradative properties in vivo. More particularly, the present invention relates to a bioabsorbable stent which has been pre-degraded prior to implantation and which still possesses sufficient mechanical properties to perform its intended function in the body, but degrades rapidly after about one to six months in vivo.

### BACKGROUND OF THE INVENTION

Intraluminal prostheses are medical devices commonly known and used in the treatment of diseased tubular organs, for example, to repair, replace or otherwise correct a defect in a tubular organ, such as a diseased blood vessel. One particular type of intraluminal prosthesis used in the repair of diseases in various body vessels is a stent. A stent is a generally longitudinal tubular device which is useful to open and support various lumens in the body, and/or provide a conduit to bypass an injured body lumen. For example, stents may be used in the vascular system, urogenital tract and bile duct, as well as in a variety of other applications in the body.

Stents are generally open-ended structures which are radially expandable between a compressed insertion diameter and an expanded implantation diameter. Stents may also be flexible in configuration, which allows them to be inserted through and conform to tortuous pathways in the blood vessel or other lumen. Such a stent is generally inserted in a radially compressed state and expanded either through a self-expanding mechanism, or through the use of balloon catheters.

One advantage of biodegradable implants and stents is that they may provide the necessary support function for a period of time, but are designed to biodegrade and be absorbed by and eventually eliminated from the body.

However, typical biodegradable materials involve the drawback that they degrade too slowly or unevenly. As a result, the implant or the stent loses strength and eventually disintegrates into small pieces and particles. The accumulation of such particles can cause temporary blockage of the lumen and can be hazardous to the patient. Longer bioerosion times can further complicate such a hazardous condition.

Accordingly, it is desirable to design a stent which maintains its mechanical properties, including strength, during the healing stage but which rapidly degrades and is quickly bioabsorbed thereafter. More particularly, it is desirable to reduce the time to onset of strength loss and total time of degradation, and also reduce the degradation rate.

### SUMMARY OF THE INVENTION

In one aspect of the invention there is provided a biodegradable implant including a biodegradable polymer implant previously exposed to conditions of biodegradation sufficient to produce greater than about a 25% reduction in mechanical properties as compared to the unexposed polymer and reduce the degradation time/rate without affecting the implant strength.

In another aspect of the invention there is provided a biodegradable implant including a stent bioeroded by chemical and/or radiation exposure. The stent possesses greater than about a 25% reduction in mechanical properties as compared to the unexposed stent.

In a further aspect of the invention there is provided a biodegradable stent which includes a braided construction of bioabsorbable filaments. Prior to implantation, the biodegradable stent is exposed to radiation in sufficient amounts to provide an initial pre-degraded state. The pre-degraded state may possess a radial compressive force of about 25% less then a non-exposed stent of the same type and construction. Additionally the biodegradable stent of the present invention may include axial runners attached to the stent to compensate for the drop in radial force properties following e-beam irradiation.

In still a further aspect of the invention there is further provided a biodegradable stent including biodegradable polymer fibers formed into a stent and having a textile construction. The stent is desirably predisposed to accelerated bioerosion through exposure to bioerosive chemicals and/or radiation prior to implantation. The stent may possess a 13% or more loss of radial compressive strength after 12 weeks of exposure.

In yet still a further aspect of invention there is provided a method for forming a biodegradable stent including the steps of irradiating a bioabsorbable fiber, forming a textile implant from the fiber, and heating the implant. The implant may possess greater then about a 25% reduction in mechanical properties as compared to a bioabsorbable implant of the same type and construction which was not exposed to radiation.

The invention provides for a biodegradable implant including a biodegradable polymer wherein only a portion of the implant is partially degraded by prior exposure to conditions. A biodegradable implant including a biodegradable polymer implant partially degraded by prior exposure to conditions of degradation sufficient to produce a strength loss of less than about 25% as compared to the polymer implant prior to exposure.

Additionally, the biodegradable implant may include two different stents, one of the different stents being positioned within the other stents, and the stents being formed of different polymer.

A biodegradable implant includes two different biodegradable polymers. The polymers have different degradable rates at least a portion of each polymer being partially degraded by prior exposure to conditions of degradation sufficient to produce a strength loss of less than about 25% as compared to the polymer implant prior to exposure.

A biodegradable implant further includes a stent located between two grafts, and only the stent is formed from biodegradable polymer.

A further aspect of the present invention includes a method of forming a biodegradable stent including the steps of forming a bioabsorbable implant from a textile construction of bioabsorbable fiber, heating the implant, irradiating the implant under sufficient amounts of radiation and for a sufficient time suitable to provide a pre-degraded implant which has lost more then about 25% of its mechanical properties as compared to the implant prior to exposure to radiation.

In yet another aspect of the present invention there is provided a method of pre-degrading a bioabsorbable implantable material which includes the steps of applying radiation to and/or bioerosive chemical(s), the material in sufficient amounts to pre-degrade the material. Desirably, the pre-degradation exposure causes chain scission in the stent material and accelerated degradation in the body.

Yet further aspect of the invention includes a method for forming a pre-degraded bioabsorbable implantable prosthesis which includes the steps of forming an implantable prosthesis from a bioabsorbable material, and irradiating the prosthesis with a beam of accelerated electrons. The bioabsorbable material exposed to the accelerated electrons desirably undergoes chain scission, which results in partial degradation and potentially loss of mechanical properties. Once implanted in the body, the stent exhibits an accelerated degradation rate as compared to its degradation without prior irradiation exposure.

### BRIEF DESCRIPTION OF THE DRAWINGS

FIG. 1 is a schematic illustration of the implant according to the present invention having a braided structure.

FIG. 2 is a schematic illustration of the implant according to the present invention having axial runners adhesively attached thereto.

FIG. 3 is a schematic illustration of the implant according to the present invention having axial runners interwoven therein.

FIG. 4 is a schematic illustration of the implant according to the present invention having a series of shortened axial runners spot attached thereto.

FIG. 5 is a graphic representation of braided stent structure made in accordance with the present invention showing losses in mechanical strength as a result of exposure to radiation and the resultant pre-degradative effect.

FIG. 6 is a partial cut-away perspective view of a stent-graft according to the present invention.

### DETAILED DESCRIPTION OF THE INVENTION

The present invention overcomes the shortcomings of the prior art by providing an implant, such as a stent, graft, stent-graft composite, or a patch, which retains its strength and mechanical properties during the healing stage of the lumen, but quickly degrades after the healing stage and may be absorbed in the body.

The implants of the present invention may be formed from a biodegradable, and desirably bioabsorbable, material which has been pre-treated to accelerate its degradation once placed in the body. In one embodiment of the invention, the implant is a radially adjustable (distensible) stent, generally formed in a tubular structure and used to support, protect or hold open a body lumen. As used herein, the term “biodegradable” and its variants refer to degradation or general breakdown of material in vivo. As used herein, the term “bioabsorbable” and its variants refer to degradation or general breakdown and metabolism of material in vivo.

Various stent types and stent constructions may be employed in the invention. Among the various stents useful include, without limitation, self-expanding stents and balloon expandable extents. The stents may be capable of radially contracting, as well and in this sense can best be described as radially distensible or deformable. Self-expanding stents include those that have a spring-like action which causes the stent to radially expand, or stents which expand due to the memory properties of the stent material for a particular configuration at a certain temperature. The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like.

Although a wide variety of distensible stent configurations may be used, one particularly useful stent configuration is a braided stent. Braided stents are known in the art. Examples of braided stents include, but are not limited to, those described in U.S. Pat. No. 4,655,771 to Hans I. Wallsten, U.S. Pat. No. 5,575,818 to Pinchuk, U.S. Pat. No. 6,083,257 to Taylor, et al., and U.S. Pat. No. 6,622,604 to Chouinard, et al, all of which are incorporated herein by reference.

Braided stents tend to be very flexible, having the ability to be placed in tortuous anatomy and still maintain patency. The flexibility of braided stents make them particularly well-suited for treating aneurysms in the aorta, where the lumen of the vessel often becomes contorted and irregular both before and after placement of the stent.

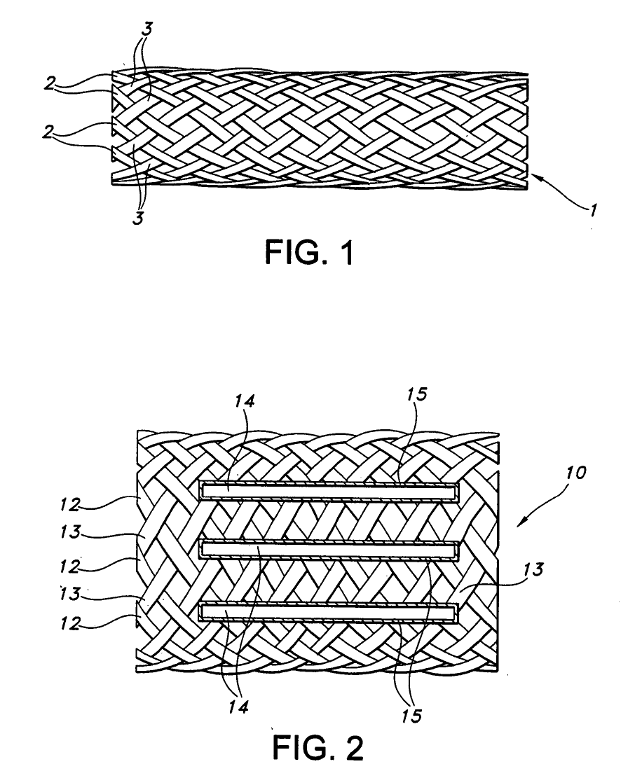

FIG. 1 shows a biodegradable braided stent structure 1 of the present invention. The braided stent structure 1 is formed of at least two continuous filaments 2, 3 which are interwoven in a pattern, thus forming overlaps. At each overlap, one filament 2 is positioned radially outward relative to the other filament 3. Following each filament along its helical path through a series of consecutive overlaps, that filament may, for example be in the radial inward position in one overlap and in the radial outward position in a next overlap, or may in the inward position for two overlaps and in the outward position for the next two, and so on. A typical braided stent is formed on a mandrel by a braiding or plaiting machine, such as a standard braiding machine known in the art and manufactured by Rotek of Ormond Beach, Fla.; Herzog and Steeger. Any suitable braiding or plaiting machine may be used, however, and the use of terminology specific to components of the machine manufactured by Rotek is not intended as a limitation to the use of that machine design.

The filaments of the implant or stent of the present invention may be multifilaments, monofilaments or spun types. In certain vascular applications, multifilaments are preferred due to the increase in flexibility. Where enhanced crush resistance is desired, the use of monofilaments has been found to be effective. Additionally, the filaments may be flat, shaped, twisted, textured, pre-shrunk or un-shrunk depending on the desired end use.

The filaments of the present invention are biodegradable polymers, copolymers and combination thereof. Suitable biodegradable polymers or copolymers include poly(alpha-hydroxy acids), such as polylactide, poly-L-lactide (PLLA), poly(D-lactide) (PDLA), poly (DL-lactide), polyglycolide (PGA) and co-polymers such as poly(L-lactide-co-D-lactide), poly(L-lactide-co-DL-lactide), poly(lactide-co-glycohide), polydioxanone, polycaprolactone, polygluconate, polylacetic acid-polyethylene oxide copolymers, modified cellulose, collagen, poly(hydroxybutyrate), polyanhydride, polyphosphoester, poly(amino acids), tyrosine-derived polyarylate, tyrosine-derived polycarbonate, poly(hydroxyvalerate) (PHV), polysaccharides, polyorthoesters, trimethylene carbonate (TMC), or related copolymers materials and combinations thereof. Further, bulk-degrading or surface-degrading polymers could be used.

Additionally, it is preferred that the filaments are also bioabsorbable to permit absorption by the body of the degradation by-products subsequent to implantation. The selection of bioabsorbable material used to form the implant depends on a number of factors, including the end-use application, the desired absorption time and the implant size and type. For example, PGA and polydioxanone are relatively fast-bioabsorbing materials (weeks to months), and PLA and polycaprolactone are relatively slow-bioabsorbing materials (months to years). Bioabsorbable polymers, such as poly(alpha-hydroxy acids) such as polylactides, polyglycolides, their co-polymers, and similar bioabsorbable polymers as known in the art, are desirable filament materials and may be employed in the present invention.

Bioabsorbable polymers, such as PLLA and PGA, degrade in vivo through hydrolytic chain scission to lacetic acid and glycolic acid, respectively, which in turn is converted to CO₂ and water and then eliminated from the body by respiration. Heterogeneous degradation of semicrystalline polymers occurs due to the fact that such materials have amorphous and crystalline regions. Degradation occurs more rapidly at amorphous regions than at crystalline regions, and strength does decrease before mass loss occurs. But the strength loss does not occur before mass loss because of the amorphous regions degrading. On the contrary, as the amorphous regions are attacked by hydrolysis there is a reduction in molecular weight without a corresponding reduction in strength because the part is still held together by it crystalline regions (bulk-degrading polymers only). Degradation time may be affected by variations in chemical composition, polymer chain structures, part geometry, loading on the implant, bulk-degrading or surface-degrading, and material processing including pre-degradation treatments, as below-discussed.

The individual filaments and/or completed implant of the present invention undergo a pre-degradation treatment prior to implantation, which imparts to the implant the ability to degrade in vivo in a significantly shorter time period. Notwithstanding the exposure to radiation, i.e., a partial degradation state, the implant retains sufficient mechanical properties and strength to serve its intended purpose during the healing stage. After the healing stage, the implant quickly degrades and is desirably absorbed by the body. Once in vivo degradation begins, the physical properties of the implant, e.g., the mechanical structure and strength, quickly degrades as the structure losses mass.

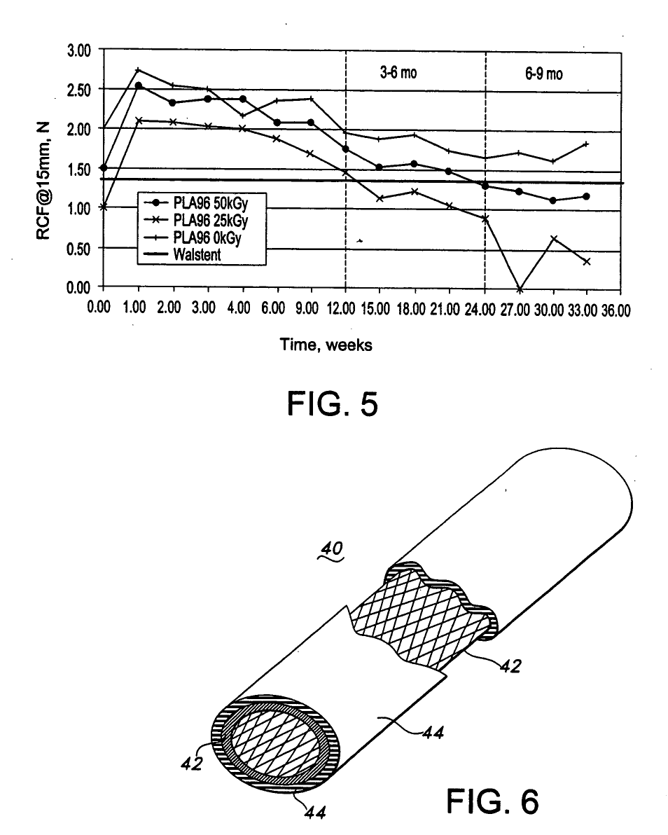

FIG. 5 shows an embodiment where the implant is a stent, the pre-degradation treatment may reduce the strength, i.e. radial compressive force, of the stent up to about 75%, preferably less than about 25% compared to an untreated implant. This means that about 25 to about 75% less force is required to radially compress the stent. The pre-degraded implant retains its physical properties sufficient to perform its intended function, i.e., mechanical structure and strength, in vivo for about 21 weeks and desirably for at least about 14 weeks in vivo. In vivo, the implant may lose its radial strength at a rate of about 0.05 N/week to about 0.08 N/week of radial compressive force. Generally, between about 9 weeks to about 24 weeks after implantation of the pre-degraded implant, the rate of degradation results in a loss of radial compressive force (RCF) of about 0.03 N/week to about 0.1 N/week; preferably, the loss rate of radial strength (RCF) is between about 0.04 N/week to about 0.1 N/week loss of RCF. After the healing stage, the loss rate of radial strength is between about 0.09 N/week to about 1.0 N/week based on loss of radial compressive force (RCF); from about 3 months to 6 months after implantation, the loss rate of RCF is about 0.1 N/week to about 0.85 N/week. FIG. 5 shows one example of the present invention of reducing the degradation time, both strength and mass degradation. Generally, the specific initial strength requirements, desired time to loss of strength, desired rate of strength loss, and desired time to full mass loss will likely be very different for different types of stents intended for different disease states (esophageal vs. cardiac vs. biliary, for example).

Radially compressive force (RCF) is the amount of force required to radially compress the stent from an expanded state or state of rest. Additionally, the radial expansive force (REF) decreases as the stent strength decreases. As the stent degrades, it's radial strength decreases and less radial force is required to radially compress the stent. Radial expansive force (REF) is the amount of outward force a stent exerts on the lumen wall.

Pre-degradation treatment to the implant may be accomplished using various techniques, including without limitation, chemical degradation, thermal degradation, radiation degradation of the individual filaments and/or completed implant and combinations thereof. One desirable degradation treatment includes exposure to radiation to yield the desired rate of degradation depending on the use. Useful radiation treatments include, but are not limited to, e-beam radiation and gamma radiation. Depending on the desired outcome the radiation treatment may occur in an inert atmosphere, such as argon gas.

Physical and/or chemical properties of polymers can be modified with radiation to provide the desired rate of degradation of the material. Included among these, are molecular weight, chain length, chain entanglement, cross-linking, polydispersity, branching, pendant functionality, chain termination, tensile strength, and elastic modulus. Generally, high-energy radiation produces ionization and excitation in polymer molecules. These energy-rich species undergo dissociation, abstraction, and addition reactions which may lead to chemical instability and a higher susceptibility to the oxidative and hydrolytic process of the body, resulting in increased biodegradable rates.

Chain scission herein is defined as a random rupturing of bonds which reduces the molecular weight, with concomitant loss in the physical properties of the material such as tensile strength and the modulus of elasticity and degrades the material. The degradation treatment of the present invention may cause chain scission of the polymer chain, dividing the chain into smaller elements, and yielding lower molecular weight polymer material and reducing the mechanical and physical properties. The smaller polymer chains allow for an accelerated degradation of the material and concomitant loss in the strength of the material, mechanical properties and mass.

The level of radiation exposure (dose) of the implant may vary depending on the desired performance and utility of the irradiated material, i.e., the length of time desired for physical and mechanical properties to be sustained in vivo and the desired rate of degradation thereafter. Dose rate refers to how fast energy is absorbed and depends on many factors including the source, strength, and size of the radiation field; its distance from the source; and the type of radiation.

The type of radiation treatment used affects the degradation and function of the implant. There are differences between electron-beam and gamma radiation treatments which are related to level of radiation exposure (dose rate) and, ultimately, to the oxidative effect of material occurring at or near the material's surface. In both gamma and electron-beam irradiation systems, available oxygen is quickly consumed within the polymer. However, in the case of electron-beam processing, the time of energy application is so short that before more oxygen can permeate into the material from its external surfaces, the application of radiant energy has been terminated. Once the radiation is terminated, direct formation of additional radicals ceases. In short exposure times, the surface of the material may undergo a greater degree of chain scission then the internal depth of the material. In gamma irradiation, the application of ionizing energy generally continues over a much longer period of time, allowing reactants, such as oxygen, to permeate into depleted areas of the material, resulting in a greater degree of oxidation and chain scission at greater depths in the material. Of course, if irradiation is done in an inert atmosphere, less strength loss occurs.

For electron and gamma sources of the same strength, the dose rate of the electron source is generally many times greater than that of the gamma source. The electron beam is usually unidirectional and is concentrated in a much smaller region, and the interaction of electrons with other electrons is much stronger than with photons of the gamma irradiation.

Additionally, the construction of the implant and characteristics of the material affects the radiation effectiveness, i.e., thickness, volume, fiber structure, molecular weight, and oxygen, moisture sensitivity, dose rate, materials of construction. Therefore the dose rate may be adjusted to accommodate these characteristics.

Exposure to radiation may be used to effectuate chain scission. During this degradative reaction, acid groups may be formed on the polymer, which serve to accelerate implant degradation. The pre-degradation treatment of the present invention may be performed at a dose rate of greater than about 25 kGy to yield chain scission of the material. It is contemplated that the dose rate may also be less than 25 kGy, depending on the desired change in degradation time/rate. Higher doses generally correspond to higher degradation rates. The dose rate may desirably range between about 27 kGy to about 100 kGy. Further, as discussed above, the type of radiation treatments greatly differ in their process and result. Therefore, the e-beam radiation pre-degradation treatment may last for seconds, while the gamma radiation pre-degradation treatment may last a few hours, to accumulate the desired dosage and degradative properties.

Irradiation may be carried out in a conventional manner, i.e. by placing the individual filaments or the completed implant in a suitable container, i.e. glass or plastic container, and exposing it to the electrons. The irradiation treatment may be performed as a single dose or multiple doses, to acquire the desired cumulative dosage which provides the desired amount of pre-degradation. The irradiation treatment could occur in an inert atmosphere or in ambient conditions. It could also occur in a variety of types of packaging (Tyvek, for example), including the product packaging if irradiation is the final processing step.

Chemical pre-degradation could occur by soaking the material or implant in a chemical bath (water, phosphate-buffered saline, or other) at either room temperature, body temperature, or higher temperatures that are at or above the material's glass transmition temperature (Tg). Thermal pre-degradation could occur by exposing the material or implant to temperatures that are above Tg for varying periods of time.

Additionally, additives can be incorporated into the polymeric material forming the implant to accelerate implant degradation and absorption. Suitable additives may hydrolyze to produce acids more rapidly than formation of acid groups on the polymer. Such additives break down in warm, wet acidic environments, so that once in vivo degradation is initiated, catalysts are generated that further accelerate degradation.

The implants of the present invention may further include support structures such as rings, struts, sutures, axial filaments and axial runners. For example, in stent embodiments, axial runners may be incorporated into the stent to enhance the radial expansion force of the implant. The runners may be incorporated into the textile construction, i.e., weave, braid or knit, etc., of the implant or are adhered to the implant thereto via thermal adhesion, chemical or mechanical adhesion. For examples, adhesives, welding or sutures may be employed. Although thread-like or suture structures are useful, this invention contemplates the use of any biocompatible, material and configuration capable of serving as constraining elements. FIGS. 2-4 each show a braided stent using different embodiments of axial runners.

The axial runners preferably are elastic i.e., they recover their shape after being stretched. The axial runners tend to counteract axially elongating forces and/or apply an axially constrictive force to the implant body. Accordingly, since axial constriction and radial expansion go hand in hand in braided stent configurations, when used in such devices, the axial runners may enhance the radial expansion and radial compression resistance of the implant. Further, the axial runners may serve to compensate to some degree for the loss in strength from the pre-degradation treatment of the implant. Further, the runners assist with creep resistance by contracting to increase the implant diameter, which may prevent or reduce unwanted elongation (and therefore decreased diameter) and subsequent migration of the implant from its intended implantation site.

The axial runners may also be fabricated of a material that shrinks in length when exposed to moisture or body temperature. The shrinkage of the axial runners will apply a longitudinally constricting and, therefore, radially expanding, force on the implant. The axial runners may be attached to the implant body such that they are in a state of non-expansion when the implant is in a quiescent state (state of rest) (i.e., the diameter when no axial or radial force is applied to the implant body). In this type of embodiment, the axial runners counteract radially constricting forces or axially elongating forces applied to the implant and thus tend to enhance the radial expansion force up to the point where the implant diameter reaches its rest position.

In one embodiment, the axial runners may be attached to the implant body such that they are in an elongated, elastomerically stretched state when the implant is at its rest diameter. In this type of embodiment, the axial runners, not only counteract radially constricting forces, as well as, axially elongating forces applied to the implant, but also increase the radial expansion force beyond the point where the implant diameter reaches what would otherwise be its rest diameter. Accordingly, the axial runners may be employed to cause the implant to have a larger diameter than it would have otherwise.

Accordingly, axial runners may be attached either by holding the implant in an axially constricted/radially expanded position during affixation of the axial runners, or by holding the axial runners in an elongated state during affixation, or both.

Suitable materials for the axial runners include non-biodegradable and bioabsorbable polymers. Desirably, bioabsorbable polymers may be employed. Polyurethane and silicone elastomers non-limiting are examples of useful biocompatible polymers. Segmented polyurethanes, such as those sold by DuPont under the trade name Lycra®, may be used. Other polyurethanes, such as those sold under the trademark Spandex® by Globe Manufacturing Corporation, may also be employed. Numerous other companies manufacture medical grade polyurethane elastomers useful in the present invention. For example, Thermedics Inc., a division of Thermo Electron Corporation, manufactures several grades of biostable polyurethane elastomers commercialized under the trade names Tecoflex®, Tecothane®, Carbothane®, Tecophilic® and Tecoplast®; Elastomedic Pty Ltd. has a family of useful polyurethane elastomers commercialized under the trade name Elast-Eon®; Cardiotech International, Inc. has a family of useful polyurethane elastomers commercialized under the trade names Chronoflex® and Chronothane®; Cardiotech International, Inc. also commercialized Chronoprene, a thermoplastic rubber elastomer that can be used to manufacture axial filaments in accordance with the present invention. The axial runners can further include a radiopaque marker material as known in the art, such as, those made from tantalum or barium sulfate.

Biodegradable polymers are particularly desirable for the axial runners. Elastomeric biodegradable polymers are particularly desirable. Useful polymeric biodegradable materials include polymers, copolymers, block polymers and combinations thereof. Among the known useful polymers or polymer classes which meet the above criteria are: poly(glycolic acid) (PGA), poly(lacetic acid) (PLA), polydioxanones, polyoxalates, poly(*-esters), polyanhydrides, PHA, and combinations thereof.

FIG. 2 illustrates a particular embodiment of the invention in which the axial runners 14 are attached to a stent structure 10 by adhesive 15. First, the axial runners 14 are brought into contact with the stent structure 10. In the particular embodiment illustrated in FIG. 2, the axial runners 14 are laid on top of the outer surface of the stent structure 10. However, the runners alternately could be laid on the inside surface of the body or interwoven into the helically wound fibers 12 and 13. Next, a suitable adhesive 15 is applied over the entire length of the axial runners 14. Application of the adhesive can be accomplished using a suitable method, for example, by syringe or glue gun. The adhesive may be in a flowable form, including in a solvent carrier, or as a hot melt which is applied in the flowable forum. The adhesive 15 may also be “spot” applied along the runners 14 to the stent structure 10. FIG. 4 shows an example of affixing the runners using “spot” applied adhesive. Stent structure 10 is then heat treated to evaporate the solvent within which the adhesive is dissolved. After the solvent has evaporated, the axial runners 14 are adhered to the stent structure 10. In the event the adhesive is applied using a solvent, the solvent may be removed by permitting evaporation or accelerated by using heat.

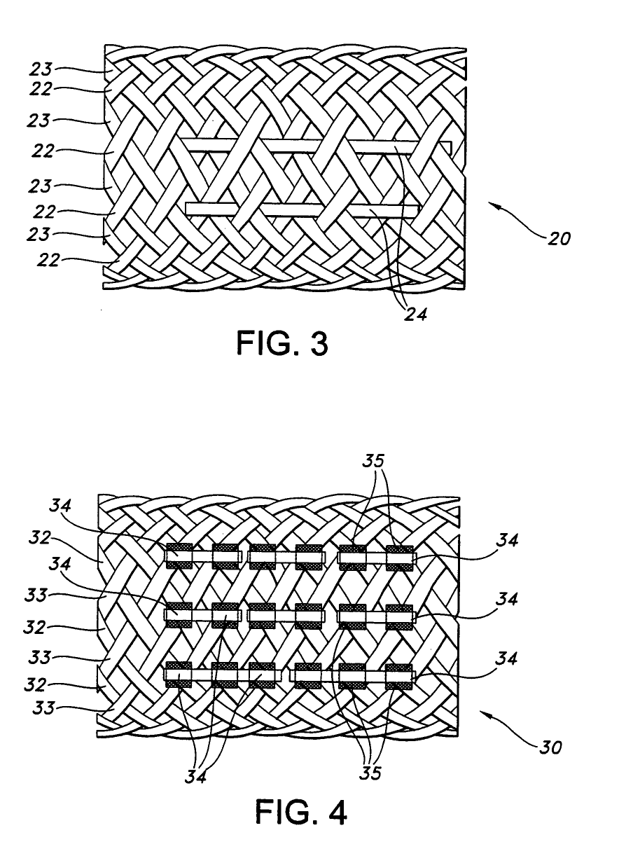

FIG. 3 shows an embodiment of the invention where axial runner 24 is interwoven with braided filaments 22, 23 of stent structure 20 at the time of the braiding of the stent structure 20. However, in alternate embodiments, axial filaments 24 could be laid on the inside surface, outside surface or on both surfaces of stent structure 20 after stent structure 20 is braided. Even further, the axial runners 24 may be woven into the stent structure 20 as shown in FIG. 3, but at a time after the stent structure 20 itself has been fully formed, and an adhesive can also be used to further secure the axial runners incorporated into the stent structure.

In yet another embodiment of the invention, the axial runner may be applied on the outer surface of the stent body, as shown in FIG. 3, the inner surface of the stent body and/or interwoven with the threads of the stent body, as shown in FIG. 3. Then the entire stent and axial runners may be sprayed, dipped or coated with the adhesive solution. The stent may then be heat treated, as is known in the art.

Further, as shown in FIG. 4, it is also contemplated that the runners be a series of short runners 34 along the stent structure 30 instead of long runners. FIG. 4 shows the short runners 34 attached to the filaments 32, 33 at various spots with an adhesive 35. Shorter runners 34 structurally break apart into smaller portions as the degradation process progresses in vivo.

The biodegradable prostheses of present invention may be coupled with a similar degradable or permanent graft, coating or additional support structure to provide additional benefits or to meet specific needs and end use applications.

In another embodiment of the present invention, the inventive implant may further include a graft member. As depicted in FIG. 6, which is a partial cut-away perspective view, stent-graft 40 includes a stent 42, which is shown as being a braided stent, and a graft member 44. Graft member 44 is depicted as covering the outer surfaces of the stent 42. The present invention, however, is not so limited. For example, the graft member may disposed over the inner surfaces of the stent to provide a liner for the stent or two graft members may be provided where one is a cover disposed over outer surfaces of the stent and the other is a liner disposed over interior stent portions. The graft 44 may fully or partially cover or line the stent 42. Any graft as known in the art may be suitable for use. Virtually any textile construction may be used for the graft, including weaves, knits, braids, filament windings, spun fibers and the like. Any weave pattern in the art, including, simple weaves, basket weaves, twill weaves, velour weaves and the like may be used. The graft structure includes a tubular member having an inner and outer wall structure and a central lumen extending from a first open end to a second open end.

Particularly useful material used for grafts intended for permanent, non-biodegrading use include, without limitation, polyesters such as polyethylene, terephthalate (PET), polyethylene, naphthalate (PEN), polytetrafluoroethylene (PTFE), polyurethanes, polysiloxanes, silicones, polyurethane/silicone copolymers and combinations of copolymers and blends thereof. Desirably, the graft may be an expanded PTFE (ePTFE) or a composite of ePTFE and textile.

The tubular grafts may be formed by techniques known in the art, including extrusion of tubes or sheets or use of various textile constructions as discussed above. When sheets are used to form the tubular graft, they are wrapped to form tubular members. Tapes may also be used and overlappingly wrapped helically to form a tubular member.

Stent/graft assemblies of the present invention may be made by covering at least a portion of one or more surfaces of the stent with graft material. Attachment of the stent to the graft may use various techniques including adhesive bonding, melting of the graft material to the stent, laminating opposing graft surfaces through the openings of the stent, suturing the graft to the stent, as well as dipping the stent into a polymeric fluid to form a graft coating, or spraying graft material on the stent.

In contrast to the biodegradable stent members of the present invention, the grafts used in combination with such stents may be made from a non-biodegradable graft material intended for permanent placement It is also contemplated, however, that the graft may be formed from biodegradable and/or bioabsorbable materials, which may be the same or different from those used for the stent. Similarly, pre-degradative processing of the graft may also be performed on the graft prior to implantation. The grafts used in the present invention may be selected from a wide variety of materials, including natural materials, modified natural materials and synthetic materials. Examples of useful synthetic materials include any of the aforementioned polymers, co-polymers, and block polymers, thereof, as well as combinations thereof.

A further aspect of the present invention relates to a method of making the inventive biodegradable implant. The method includes the steps of providing a biodegradable filament, pre-degrading the filament, and forming an implant therefrom. The formation of the implant may be accomplished using a textile construction or an extrusion method. Alternative methods include providing a biodegradable filament, forming an implant using a textile construction, such as a braid, a weave or a knit, from the biodegradable filament, and pre-degrading the formed implant by subjecting it to radiation as described herein. Additional steps may include the incorporation of axial runners, coatings, and attachment of grafts.

The filaments for forming the biodegradable and preferably bioabsorbable implant may be extruded and drawn. The filaments may then be subjected to radiation to provide the pre-degraded properties desired and then formed into an implantable structure, or first formed into a structure suitable for implantation, such as a stent, and subsequently exposed to radiation to obtain the desired pre-degraded structure.

The pre-degradation step includes exposing the individual filaments or completed implant to a predegradation treatment using chemical, thermal or radiation sources. A combination of degradative sources may be employed. The preferred method of degradation treatment is exposure to radiation for a pre-determined dosage and/or time. Radiation exposure includes e-beam exposure or gamma beam exposure may be up to 100 kGy, preferably between about 25 kGy to about 100 kGy (kilogray), more preferably about 40 kGy to about 70 kGy. The exposure to gamma beam radiation may take several hours, depending on the dosage requirements, while the e-beam may take only a few seconds. Generally, the individual filaments or formed implant is exposed to enough radiation to permit molecular chain break-down and create smaller polymer unites or molecular chains which allow for quicker degradation and for absorbability in vivo. Radiation exposure reduces the molecular weight of the polymeric material, allowing for quicker degradation in vivo. However, as discussed above, the implants of the present invention are formed by selecting the appropriate materials and degradative exposure conditions such that the implant retains sufficient structural integrity, strength and physical properties to serve its intended purpose in vivo, yet rapidly degrade once its useful period has expired. Generally, the implants of the present invention are designed to maintain their useful properties through the healing period, which may be up to about 5-6 months. However, the time frame changes depending on the end use of the implant—both the intended location of the implant and the disease state that is to be treated. Soft tissue healing is typically 12-15 weeks, or closer to 3-4 months. For example, the choice of material and dose exposure to radiation may be designed to provide adequate physical properties, i.e., mechanical strength, shape and structural integrity during the time of deployment and for about 21 weeks once implanted, preferably between about 9 weeks to about 15 weeks, through the healing phase. After the healing stage, the material rapidly degrades, thereby decreasing in strength and physical properties and eventually results in mass loss.

In order to deliver the biodegradable implant or stent of the invention to the site intended in vivo, the external diameter of the implant may be reduced to permit introduction through the blood lumen and deployment at the targeted site. In the case of braided stents, the implant may be reduced by, for example, elongating the implant, to allow for a corresponding reduction in diameter, and maintaining the reduced diameter during the delivery process. Once at the targeted portion of the body lumen, the stent is expanded or permitted to expand and thus deployed within the body lumen.

As described above, a further aspect of the present invention relates to a method of making a composite stent/graft. The method includes the steps of providing a pre-degraded implant, as above discussed, and attaching a graft member using conventional techniques. Alternatively, it is contemplated that the stent-graft composite is formed prior to the pre-degradation treatment, and the completed composite undergoes the pre-degradation treatment, as above-discussed.

Further, the present invention contemplates a method of forming a biodegradable implant having a bioabsorbable coating thereon. The implant is placed over the mandrel and the coating is applied thereto. In particular, the biodegradable coating layer may be applied as a fluid coating-material on the surface(s) of the implant by such means as dipping, spraying or painting. The biodegradable coating may be applied in a single layer or in multiple layers. A bioactive agent may be incorporated within the biodegradable coating material.

Also, the stent 1 may be treated with any known or useful bioactive agent or drug including without limitation the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promotors (such as growth factor inhibitors, growth factor receptor antagonists, transcriptional activators, and translational promotors); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

Further, the present invention includes a method of forming a prosthesis including the steps of providing a pre-degraded implant as above discussed, providing a support structure attached thereto. The preferred support structure includes axial runners. The axial runners may be attached to the implant via mechanical, thermal or chemical mechanisms. The axial runners may be attached by weaving them within the textile construction of the implant or thermal or mechanical attachment, as above discussed. The axial runners may be attached after the implant has undergone the pre-degradation treatment. Alternatively, the axial runners may be attached to the implant and the entire structure is subjected to pre-degradation treatment.

### EXAMPLES

### Example 1

A stent in accordance with the present invention was manufactured with 36 braided threads of a diameter of 0.5 mm. The threads were made of a copolymer of 96% poly-L-lactide and 4% poly-D-lactide (PLA96). The stent was formed having an initial fully opened diameter of about 22 mm. The unexposed PLA96 stent in the example has Radial Compressive Force˜2.00 N when compressed at a 15 mm diameter, according to the graph in FIG. 5. The graph really only shows the difference in RCF at 15 mm between the three groups (unexposed, 25 kGy, and 50 kGy) initially and over time, as tested by a specific test method. The stent structure was exposed to e-beam radiation at various intensities to yield a predetermined dosage. FIG. 5 is a graft showing the loss of mechanical strength of pre-degraded stent structures at various levels of radiation exposure, 0 kGy, 25 kGy and 50 kGy. The pre-degraded stent structures were tested for the strength of the radial force of the pre-degraded stent at various points in time. Finished stents were loaded into the stent delivery systems, held at 37 C for 10 minutes to simulate stent placement, deployed, and were radial force tested at initial time point (time=0). The radial force test is performed by placing a collar around the stent and compressing the stent to a diameter of about 15 mm. These stents are then placed in 7.4 pH phosphate buffered saline solution (PBS) at 37 C to stimulate in vivo blood conditions. At each time interval, stents were removed from the PBS solution, radial force tested again, and then placed back into the PBS solution until the next test. FIG. 5 shows the radial force test results of each of the pre-degraded stent structures, as well as a similarly formed non-pre-degraded metal stent having a constant strength of about 1.35 N. As shown in FIG. 5, the e-beam radiation exposure resulted in decrease in the time to strength loss and eventual stent fracture as compared to stents that had not been exposed to radiation. The higher the radiation exposure level of the stent, the faster the degradation rate and ultimate stent structural breakdown.

### Example 2

A stent in accordance with the present invention was manufactured with 36 braided threads of a diameter of 0.5 mm. The threads were made of poly(L-lactide) (PLLA). The stent structure was exposed to about 50 kGy e-beam radiation, in a manner as above discussed in Example 1. Four elastomeric runners made from medical grade thermoplastic polyurethane sold commercially under the trade name Tecoflex 80-A, by Thermedics Polymer Products, Wilmington, Mass., having a diameter of approximately 0.25 mm each were attached to the stent structure. The axial runners were adhered to the stent by applying an adhesive MADE from Tecoflex 80-A dissolved in methylene chloride.

While the invention has been described in relation to the preferred embodiments withexamples, it will be understood by those skilled in the art that various changes may be made without deviating from the spirit and scope of the invention as defined in the appended claims.

What is claimed is:

1. A biodegradable implant comprising a biodegradable polymer implant partially degraded by prior exposure to conditions of degradation sufficient to produce a strength loss of less than about 25% as compared to the polymer implant prior to exposure.

2. The biodegradable implant of claim 1, wherein said implant comprises a bioabsorbable material selected from a group consisting of poly(alpha-hydroxy acid), polylactide, poly-L-lactide (PLLA), poly(D-lactide) (PDLA), poly(DL-lactide), polygylycolide (PGA), poly(L-lactide-co-D-lactide), poly(L-lactide-co-DL-lactide), poly(lactide-co-glycohide), polydioxanone, polycaprolactone, polygluconate, polylacetic acid-polyethylene oxide copolymers, modified cellulose, collagen, poly(hydroxybutyrate), polyanhydride, polyphosphoester, poly(amino acids), tyrosine-derived polyarylate, tyrosine-derived polycarbonate, poly(hydroxyvalerate) (PHV), polysaccharides and combinations thereof.

3. The biodegradable implant of claim 1, wherein said implant comprises a textile structure selected from a group consisting of a knitted textile structure, a woven textile structure, a braided textile structure, a non-woven spun structure and combinations thereof.

4. The biodegradable implant of claim 3, further including support structures selected from a group consisting of rings, struts, sutures, axial filaments, axial runners, and combination thereof.

5. The biodegradable implant of claim 4, wherein said support structure comprises a series of biodegradable axial runners.

6. The biodegradable implant of claim 1, wherein said conditions of biodegradation comprises exposure to radiation in sufficient amounts to cause chain scission of said polymer.

7. The biodegradable implant of claim 6, wherein the radiation exposure comprises e-beam radiation at a dose rate of about 25 kGy to about 100 kGy.

8. The biodegradable implant of claim 6, wherein said radiation exposure comprises gamma radiation at a dose rate of about 25 kGy to about 100 kGy.

9. A biodegradable implant comprising a stent bioeroded by chemical and/or radiation exposure, which stent shows a less than about 25% reduction in strength as compared to the stent implant prior to exposure.

10. A biodegradable stent comprising:
biodegradable stent having a braided construction of bioabsorbable filaments, said stent being exposed to radiation in sufficient amounts to provide an initial pre-degraded structure, said pre-degraded structure comprising a radial compressive strength of at least about 25% less then the stent prior to predegradation.

11. The biodegradable stent of claim 10, wherein said radiation in sufficient amounts comprises about 25 kGy to about 75 kGy of e-beam radiation.

12. The biodegradable stent of claim 10, wherein said radiation in sufficient amounts comprises about 25 kGy to about 75 kGy of gamma radiation.

13. A method of forming a biodegradable stent comprising the steps of:
irradiating bioabsorbable fibers;
forming a textile implant from said fibers, said implant having a less than about 25% reduction in strength as compared to the bioabsorbable implant prior to exposure to radiation; and
heating treating said implant.

14. The method of claim 13, wherein said step of electron beam has an energy of about 25 to about 80 kGy.

15. The method of claim 13, wherein said step of irradiating is exposure to gamma beam radiation.

16. A method of forming a biodegradable stent comprising the steps of:
forming a bioabsorbable implant from a textile construction of bioabsorbable fibers;
heating treating said implant;
irradiating said implant in an amount suitable to provide a pre-degraded implant having a less then about 25% reduction in mechanical properties as compared to the implant prior to exposure to radiation.

17. A method of pre-degrading a bioabsorbable implantable material, comprising exposing said implantable material to an electron beam radiation does of about 25 KGy to about 75 KGy for a time period sufficient to cause degradation.

18. A method for forming pre-degraded bioabsorbable implantable prosthesis comprising the steps of:
forming an implantable prosthesis from a bioabsorbable material comprising polymeric chains; and
irradiating said prosthesis with a beam of accelerated electrons, for a time sufficient to cause scission of the polymeric chains.

19. A method for forming pre-degraded bioabsorbable stent comprising the steps of:
forming a braided stent from a polylactide polymer; and
irradiating said stent with electron beam radiation between about 25 kGy to about 70 kGy, wherein said stent maintains its physical structure but exhibits a loss of mechanical properties.

20. A biodegradable implant comprising a biodegradable polymer wherein only a portion of the implant is partially degraded by prior exposure to conditions of degradation sufficient to produce a strength loss of less than about 25% as compared to the polymer implant prior to exposure.

21. A biodegradable implant comprising of claim 20, wherein said implant comprises a stent located between two grafts, only said stent is formed from said biodegradable polymer.

22. A biodegradable implant comprising two different biodegradable polymers, said polymers having different degradable rates at least a portion of each polymer being partially degraded by prior exposure to conditions of degradation sufficient to produce a strength loss of less than about 25% as compared to the polymer implant prior to exposure.

23. A biodegradable implant comprising of claim 22, wherein said implant comprises two different stents, one of said different stent being positioned within other of said different stent, said stent being formed of said different polymer.
